(19) United States
(12) Patent Application Publication
(10) Pub. No.: US 2020/0060948 A1
(43) Pub. Date: Feb. 27, 2020

(54) BRUSH-APPLICABLE POWDER NAIL POLISH AND METHODS OF MANUFACTURING AND APPLYING THE SAME

(71) Applicant: Teri Lea Zaite, Loxahatchee, FL (US)

(72) Inventor: Teri Lea Zaite, Loxahatchee, FL (US)

(21) Appl. No.: 16/670,089

(22) Filed: Oct. 31, 2019

Related U.S. Application Data
(63) Continuation of application No. 15/725,565, filed on Oct. 5, 2017.
(60) Provisional application No. 62/411,395, filed on Oct. 21, 2016.

(51) Int. Cl. A61K 8/02; A61Q 3/02; A61K 8/26; A61K 8/36; A61K 8/64; A61K 8/67; A45D 29/12; A61K 8/98

(52) U.S. Cl. CPC A61K 8/022; A61Q 3/02; A61K 8/26; A61K 8/361; A61K 8/64; A61K 8/678; A45D 33/006; A61K 8/987; A61K 2800/43; A61K 2800/805; A61K 2800/30; A61K 2800/87; A61K 2800/81; A45D 29/12

(57) ABSTRACT

A powder nail polish admixture and a method of manufacturing and using the same is provided. The powder nail polish admixture has a plurality of base components including a portion of kaolin clay; a portion of sericite mica; a portion of magnesium myristate; a portion of micro fine silk powder; a portion of delaminated talc; and a portion of mixed tocopherols in powder form, wherein the plurality of base components is appropriately weighted and mixed through tumbling. In application, the powder nail polish admixture is painted onto a base coat, thereafter a separate clear top coat is painting thereon, and cured with LED lighting.

### CROSS-REFERENCE TO RELATED APPLICATION

This application claims the benefit of priority of U.S. provisional application No. 62/411,395, filed 21 OCT. 2016, and this application claims the benefit of priority as a continuation of U.S. nonprovisional application Ser. No. 15/725,565, filed 5 OCT. 2017, the contents of which are herein incorporated by reference.

### BACKGROUND OF THE INVENTION

The present invention relates to nail polish and, more particularly, to a customizable powder nail polish and methods of manufacturing and applying the same.

Current methods for applying liquid nail polish can be difficult, frustrating and messy especially for an individual user using the non-dominant hand to make the application. Users who apply liquid nail polish frequently complain about getting the polish on the cuticles or sides of fingers, which in turn have to be cleaned off with polish remover. Not only does having to remove the mess of excess nail polish involve additional steps, but frequently the excess nail polish results in a difficult to remove stain. Users of such nail polish methods also have to wait for it to dry, which often leads to nicks and smudges.

Currently dip nail polishes systems require users to dip their entire finger in resins, polymers or plasticizers powder which also leads to spills as well as stains on the fingers from the pigments. Dip nail polish systems of today also require a buffer or grinder to remove the polish which can damage the natural nail bed. Furthermore, dip polishes involve a five-or-more step process which takes a lot of time.

As can be seen, there is a need for a method of applying a unique nail polish that greatly reduces application time, does not require clean-up of polish, is powder dry so as to require only 30 seconds after applying a top coat to dry under an LED. By painting fingernails through brushing with a powdered nail polish adapted to only adhere to a base coat, and not the rest of the finger and cuticles, the present invention overcomes the disadvantages of the prior art in three-step process which can be completed in minutes, and quickly dried so as to lessens the chance of ruining the resulting application of polish. Furthermore, the ability to customize and create your own custom color by mixing two or more existing colors creates a unique experience for the user.

### SUMMARY OF THE INVENTION

In one aspect of the present invention, a powdered nail polish admixture applicable by a brush includes a plurality of base components including: a portion of kaolin clay; a portion of coated and uncoated sericite mica; a portion of magnesium myristate; a portion of micro fine silk powder; a portion of delaminated talc and a portion of mixed tocopherols in powder form.

In another aspect of the present invention, a method of manufacturing the powdered nail polish admixture includes the step of mixing the plurality of base components through gentle tumbling for approximately five minutes.

In yet another aspect of the present invention, the method of applying the powder nail polish admixture includes providing a brush applicator; loading the brush applicator with said resulting admixture; and brushing said resulting admixture onto a clear base coat.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention;

FIG. 2 is a perspective view of an exemplary embodiment of the present invention; and

FIG. 3 is a perspective view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a powder nail polish admixture and a method of manufacturing and using the same. The powder nail polish admixture has a plurality of base components including a portion of kaolin clay; a portion of sericite mica; a portion of magnesium myristate; a portion of micro fine silk powder; a portion of delaminated talc; and a portion of mixed tocopherols in powder form, wherein the plurality of base components is appropriately weighted and mixed through tumbling. In application, the powder nail polish admixture is painted onto a base coat, thereafter a separate clear top coat is painting thereon, and cured with LED lighting.

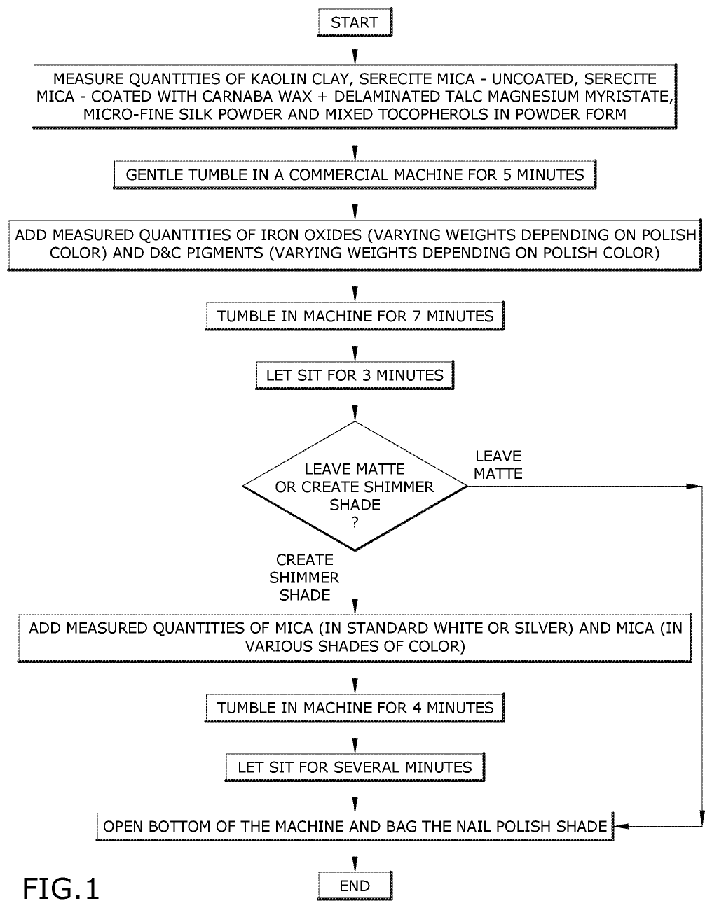

Referring to FIGS. 1 through 3, the present invention may include a polish admixture 16 taking powder form and including of a plurality of the following components: iron oxide, D&C pigments (such as FDA approved color additives for us in cosmetics), mica, kaolin clay, sericite mica, magnesium myristate, micro fine silk powder, delaminated talc, and mixed tocopherols in powder form. The iron oxide and D&C pigments may vary in weight depending on the polish color. The sericite mica may be uncoated or coated with carnaba wax. The micas may be in white, silver, or various shades of color depending on the color of a desired polish admixture 16.

Referring to FIG. 1, a method of manufacturing the polish admixture 16 may include mixing at least one of the iron oxide and D&C pigments together with the kaolin clay for adhesion, with sericite mica for slip, and the magnesium myristate as a non-whitening binder and additional adhesion. The delaminated talc may be added for translucency which maintains true and bright colors. The micro fine silk powder may be added for a smooth silky texture, while the mixed tocopherols in powder form may be mixed in as a preservative for a matte nail polish color. The micas may be added for a shimmer nail polish color. Wherein the plurality of base components further does not include a portion of the following: a resin, a polymer, an acrylic, a plasticizer base, a nail hardening agent or a liquid adhesive.

The kaolin clay, sericite mica, magnesium myristate, micro fine silk powder, and mixed tocopherols in powder form may be considered the base ingredients whose combination requires appropriate weighted portions and mixture through gentle tumbling, for example in a commercial tumbling machine for approximately five minutes. In certain embodiments, the ratios for the individual base ingredients to the plurality of base components may be approximately as follows: magnesium myristate at 32.5:100 (plurality of base components) by percentage or 226:695 by weight; kaolin at 29:100 by percentage or 202:695 by weight; uncoated sericite mica at 21.6:100 by percentage or 150:695 by weight; coated (carnauba wax) sericite mica at 7.2:100 by percentage or 50:695 by weight; delaminated talc at 5.7:100 by percentage or 26:695 by weight; micro fine silk powder at 3.7:100 by percentage or 26:695 by weight; and tocopherol at 0.3:100 by percentage or 2:695 by weight. Pigment rations range from 1:1 to 1:5 per admixture depending on the number of shades used to create a color.

Then the base ingredients may be set aside. An individual may continue to manufacture the polish admixture 16 by weighing the pigments (iron oxide and D&C pigments, varying the weights of the pigments depending on polish color) then add them to the tumbling machine. The resulting base ingredients and pigments may be tumbled for approximately seven minutes. Then the individual may allow it to sit for approximately three minutes to allow the dust plume to dissipate before opening. Then the individual may open the output end of the tumbling machine and bag the finished matte shade.

Too create a shimmer shade, the individual may perform all above steps and then carefully weigh the micas (in standard white or silver and/or in various shades of color) and add to the input end of the tumbling machine. Tumble for an additional four minutes, allowing the ingredients to sit for a few more minutes to dissipate the dust plume. Open the output end of the tumbling machine and bag the nail polish shade.

A method of using the present invention may include the following. The polish admixture 16 disclosed above may be provided. The polish admixture 16 forms a powder that can be applied for eliminating all the difficulties that traditional liquid and dip systems pose. The user may will simply paint a fingernail with a clear base coat and then cure it under LED light for 30 seconds. Then the user may load a brush 10 with the powder polish admixture 16 and brush it over the clear base coat starting from back or front. Last the user may paint the clear top coat and cure under an LED light for 30 seconds. Because the powder polish admixture 16 is a dry powder, accidentally smudging or nicking of the resulting application is impossible. And because the powder only adheres to the tacky base coat none will remain on your fingers so clean up is eliminated. The customizable powder polish makes it easy for a user to be creative and paint multiple colors and/or unique designs. The ability of the user to customize the colors distinguishes the present invention from what is currently available in liquid form, wherein the color is predetermined.

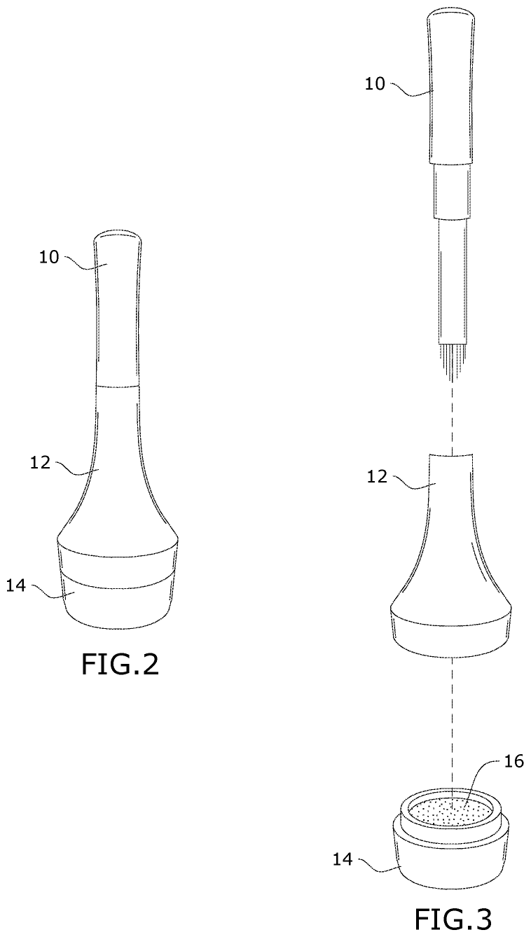

The powder polish admixture 16 may be stored in the base portion of a bottle 12 adapted to operatively associate with the brush 10 before, during, and after application, as illustrated in FIGS. 2 and 3. Additionally, the powder polish admixture 16 may also be used to paint other objects, such as figurines, crafts, or plastic or metallic products.

It should be understood, of course, that the foregoing relates to exemplary embodiments of the invention and that modifications may be made without departing from the spirit and scope of the invention as set forth in the following claims.

What is claimed is:

1. A method of applying a nail polish, comprising:
providing a powder nail polish resulting from mixing a portion of kaolin clay, a portion of magnesium myristate, and a portion of micro fine silk powder;
providing a brush applicator;
applying a base coat to a surface; and
brushing said powder nail polish by way of the brush applicator onto the base coat.

2. The method of claim 1, further comprising:
brushing a clear top coat over the powder nail polish; and curing the clear top coat under an LED light.

3. The method of claim 1, the powder nail polish further includes a portion of sericite mica.

4. The method of claim 1, the powder nail polish further includes a portion of mixed tocopherols in powder form.

5. The method of claim 1, wherein the base coat is tacky upon brushing said powder nail polish.

6. The method of claim 1, further including curing the base coat under an LED light until the base coat is tacky.

7. A nail polished surface comprising particles, the particles comprising a portion of kaolin clay, a portion of magnesium myristate, and a portion of micro fine silk powder, said particles being coated by a brush in a powdered form directly on a base coat applied to a surface.

8. The nail polished surface of claim 7, further comprising:
a clear top coat brushed over the particles and curing the clear top coat under an LED light.

9. The nail polished surface of claim 7, the particles further comprise a portion of sericite mica.

10. The nail polished surface of claim 7, the particles further comprise a portion of mixed tocopherols in powder form.

11. The nail polished surface of claim 7, wherein the nail polished surface is matte.
